(19) United States
(12) Patent Application Publication
(10) Pub. No.: US 2010/0331698 A1
(43) Pub. Date: Dec. 30, 2010

(54) ULTRASONIC DIAGNOSTIC APPARATUS, ULTRASONIC ELASTICITY INFORMATION PROCESSING METHOD AND ULTRASONIC ELASTICITY INFORMATION PROCESSING PROGRAM

(76) Inventor: Akiko Tonomura, Tokyo (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 12/918,228

(22) PCT Filed: Feb. 16, 2009
(86) PCT No.: PCT/JP2009/052461

§ 371 Date: Aug. 18, 2010

(30) Foreign Application Priority Data
Feb. 18, 2008 (JP) 2008-036541

(51) Int. Cl. A61B 8/14

(52) U.S. Cl. 600/443

(57) ABSTRACT

An ultrasonic diagnostic apparatus characterized by comprising: an ultrasonic probe for transmitting/receiving an ultrasonic wave to/from an object; a phasing and summing unit for generating RF signal frame data of a cross-sectional site of the object on the basis of a reflection echo signal measured by the ultrasonic probe; an elasticity calculator for generating frame data of elasticity information representing the degree of hardness or softness of a tissue at each measurement point of the cross-sectional site on the basis of a pair of RF signal frame data acquired in different times; a weighting unit for weighting the elasticity information of each measurement point of the generated elasticity frame data in accordance with the degree of sharpness of a distribution of elasticity information for plural measurement points containing adjacent measurement points; a smoothing unit for smoothing the weighted elasticity information of the plurality of elasticity frame data generated at the different times; and an elasticity image display unit for generating an elasticity image on the basis of the smoothed elasticity frame data and displaying the generated elasticity image on a display unit.

### TECHNICAL FIELD

The present invention relates to an ultrasonic diagnostic apparatus, an ultrasonic elasticity information processing method and an ultrasonic elasticity information processing program, and particularly to a technique of enhancing image quality of an elasticity image generated by obtaining elasticity information representing hardness or softness of a biometric tissue of a cross-sectional site from a pair of RF signal frame data acquired at different times.

### BACKGROUND ART

A conventional general ultrasonic diagnostic apparatus generates RF signal frame data on the basis of a reflection echo signal obtained by receiving/transmitting an ultrasonic wave from/to an object to be examined through an ultrasonic probe, and displays the structure of a biometric tissue of a cross-sectional site of the object on the basis of the RF signal frame data as a tomographic image such as a B mode image, for example.

Furthermore, it has been recently adopted that an elasticity image representing hardness or softness of a biometric tissue is generated on the basis of a pair of RF signal frame data which are generated and acquired at different times while an object is pressed by an ultrasonic probe according to a manual or mechanical method. That is, a displacement of a biometric tissue caused by pressing, distortion caused by the displacement and elasticity information representing hardness or softness of the tissue such as elasticity modulus are obtained every measurement point of a cross-sectional site to generate elasticity frame data, and an elasticity image is generated on the basis of the generated elasticity frame data.

With respect to the elasticity image as described above, it is known that an elasticity image is stably generated by adding plural elasticity frame data as described in Patent Document 1.

Patent Document 1: U.S. Pat. No. 6,558,324

### DISCLOSURE OF THE INVENTION

### Problem to be Solved by the Invention

However, the technique described in the patent document 1 merely directly smoothes the elasticity information of each measurement point of the elasticity frame data, and room for enhancing the image quality of the smoothed elasticity image is left.

That is, for example, in a blood stream area, a tissue is displaced due to a factor other than the press of the probe, and thus obtained displacement information and elasticity information based on the displacement information contain information (noise) other than the displacement information of the tissue which is caused by the press of the probe. Accordingly, it is hard to say that elasticity information obtained in an area where a tissue is displaced due to a factor other than the press of the probe properly reflects hardness or softness of the tissue in the area.

When elasticity frame data containing elasticity information in the noise area as described above are smoothed, the smoothed elasticity frame data directly reflect the improper elasticity information of the noise area, and thus it may adversely affect the image quality of the elasticity image.

Therefore, the present invention has an object to enhance the image quality of an elasticity image obtained by smoothing plural elasticity frame data which are generated at different times.

### Means of Solving the Problem

In order to attain the above object, according to the present invention, elasticity information of a plurality of elasticity frame data generated at different times is smoothed, and an elasticity image is generated on the basis of the smoothed elasticity frame data to enhance the image quality of the elasticity image. Preferably, the elasticity information of the plurality of elasticity frame data generated at different times which are weighted is smoothed on the basis of the weighting degree of the elasticity information of each measurement point.

Specifically, the present invention is characterized by comprising: an ultrasonic probe for transmitting/receiving an ultrasonic wave to/from an object; a phasing and summing unit for generating RF signal frame data of a cross-sectional site of the object on the basis of a reflection echo signal measured by the ultrasonic probe; an elasticity calculator for generating frame data of elasticity information representing the degree of hardness or softness of a tissue at each measurement point of the cross-sectional site on the basis of a pair of RF signal frame data acquired in different times; a weighting unit for weighting the elasticity information of each measurement point of the generated elasticity frame data in accordance with sharpness degree of a distribution of elasticity information for plural measurement points containing adjacent measurement points; a smoothing unit for smoothing the elasticity information of the plurality of weighted elasticity frame data generated at the different times; and an elasticity image display unit for generating an elasticity image on the basis of the smoothed elasticity frame data and displaying the generated elasticity image on a display unit.

Furthermore, an ultrasonic elasticity information processing method for smoothing, between elasticity frame data generated at different times, frame data of elasticity information representing the degree of hardness or softness of a measurement point of a tissue at the measurement point of a cross-sectional site of an object which is generated from a pair of RF signal frame data acquired at different times based on reflection echo signals of the cross-sectional site of the object measured by transmitting/receiving ultrasonic waves to/from the object and displaying the smoothed frame data on a display unit is characterized by comprising: a step of weighting elasticity information of each measurement point of the generated elasticity frame data in accordance with sharpness degree of distribution of elasticity information of plural measurement points containing adjacent measurement points; and a step of smoothing elasticity information of plural weighted elasticity frame data generated at different times.

Furthermore, an ultrasonic elasticity information processing program for smoothing, between elasticity frame data generated at different times, frame data of elasticity information representing the degree of hardness or softness of a measurement point of a tissue at the measurement point of a cross-sectional site of an object which is generated from a pair of RF signal frame data acquired at different times based on reflection echo signals of the cross-sectional site of the object measured by transmitting/receiving ultrasonic waves to/from the object and displaying the smoothed frame data on a display unit is characterized by containing a step of weighting elasticity information of each measurement point of the generated elasticity data in accordance with sharpness degree of distribution of elasticity information of plural measurement points containing adjacent measurement points and smoothing elasticity information of plural weighted elasticity frame data generated at different times.

### Effect of the Invention

As described above, according to the ultrasonic diagnostic apparatus, the ultrasonic elasticity information processing method and the ultrasonic elasticity information processing program of the present invention, the image quality of an elasticity image obtained by smoothing plural elasticity frame data generated at different times can be enhanced.

### BRIEF DESCRIPTION OF THE DRAWINGS

FIG. 1 is a diagram showing the overall construction of an ultrasonic diagnostic apparatus according to the present invention.

FIG. 2 is a diagram showing the detailed construction of a noise area detecting unit, a noise removing processing unit, a smoothing processing unit, etc.

FIG. 3 is a diagram showing processing concept when a smoothing processing unit is an averaging filter.

FIG. 4 is a diagram showing the detailed construction of a color scan converter.

### DESCRIPTION OF REFERENCE NUMERALS

10 ultrasonic diagnostic apparatus, 12 ultrasonic probe, 20 phasing and summing circuit, 24 image display unit, displacement measuring unit, 32 distortion amount and elasticity modulus calculating circuit, 34 noise area detecting unit, 36 noise removing processing unit, 38 smoothing processing unit

### MODES FOR CARRYING OUT THE INVENTION

An embodiment of an ultrasonic diagnostic apparatus to which the present invention is applied will be described. In the following description, the parts having the same functions are represented by the same reference numerals, and the duplicative description thereof is omitted.

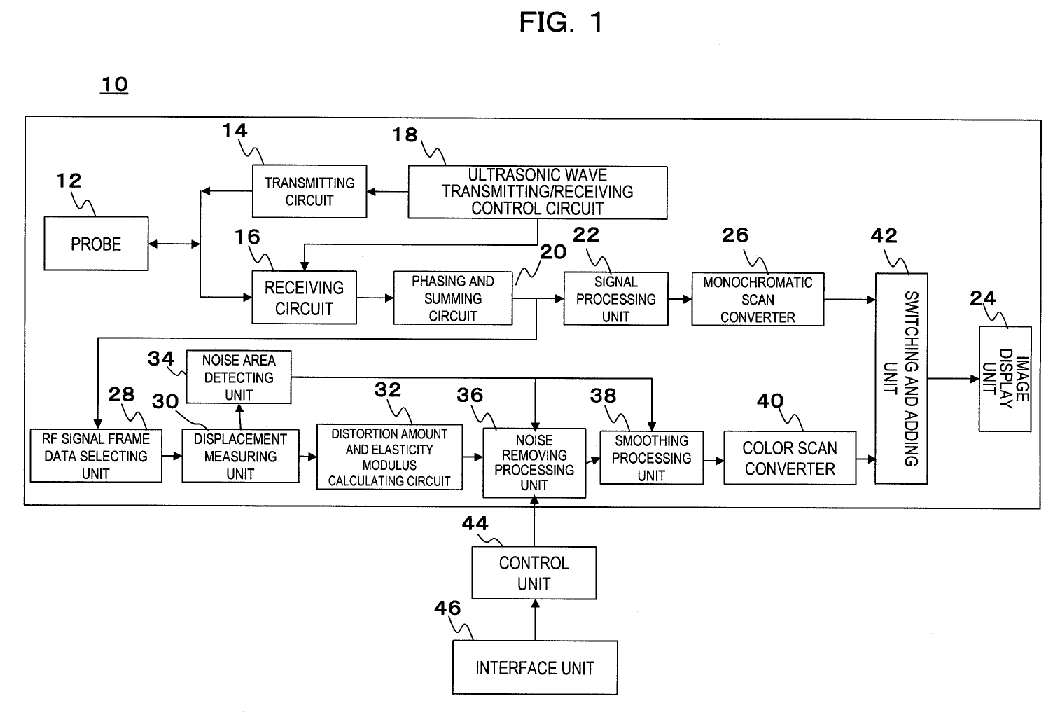

FIG. 1 is a block diagram showing the overall construction of the ultrasonic diagnostic apparatus according to the embodiment. The ultrasonic diagnostic apparatus obtains a tomographic image of a diagnosis site of an object by using ultrasonic waves, and displays an elasticity image representing hardness or softness of a biometric tissue.

As shown in FIG. 1, the ultrasonic diagnostic apparatus 10 has an ultrasonic probe 12 which is used in contact with an object, a transmitting circuit 14 for repetitively transmitting ultrasonic waves to the object through the ultrasonic probe 12 at a time interval, a receiving circuit 16 for receiving time-series reflection echo signals generated from the object, an ultrasonic wave transmitting/receiving control circuit 18 for controlling the transmitting circuit 14 and the receiving circuit 16, and a phasing and summing circuit 20 for phasing and summing reflection echoes received by the receiving circuit 16.

Furthermore, there are provided a signal processing unit 22 for constructing a grayscale tomographic image such as a monochromatic tomographic image of an object on the basis of RF signal frame data from the phasing and summing circuit 20, and a monochromatic scan converter 26 for converting an output signal of the signal processing unit 22 so that the output signal is matched with display of the image display unit 24.

Still furthermore, there are also provided an RF signal frame data selecting unit 28 for storing RF signal frame data output from the phasing and summing circuit 20 and selecting frame data of at least two frames, a displacement measuring unit 30 for measuring a displacement of a biometric tissue of an object, and a distortion amount and elasticity modulus calculating circuit 32 for determining elasticity information such as distortion, elasticity modulus from the displacement information measured by the displacement measuring unit 30.

Furthermore, a noise area detecting unit 34 for detecting a noise area of displacement frame data on the basis of the displacement frame data output from the displacement measuring unit 30, a noise removing processing unit 36 for removing the noise area detected by the noise area detecting unit 34 out of the elasticity information of the elasticity frame data output from the distortion amount and elasticity modulus calculating circuit 32, and a smoothing processing unit 38 for smoothing the elasticity information of plural elasticity frame data generated at different times which are subjected to noise removing processing are provided as a feature portion of the ultrasonic diagnostic apparatus of this embodiment. The details of these units will be described later.

Furthermore, a color scan converter 40 for performing conversion on the basis of the smoothed elasticity frame data so as to be matched with the display of the image display unit 24, and a switching and adding unit 42 for adding or switching monochromatic tomographic image data output from the monochromatic scan converter 26 and elasticity image data output from the color scan converter 40 are provided. Furthermore, a controller 44 for outputting various kinds of control signals to respective units constituting the ultrasonic diagnostic apparatus, and an interface unit 46 such as a keyboard for receiving an instruction from an examiner and outputting a command to the controller 44 are provided.

The respective units of the ultrasonic diagnostic apparatus 10 will be described hereunder in detail.

The ultrasonic probe 12 is formed by arranging many transducers in a strip-like form, and it transmits/receives ultrasonic waves to/from an object by mechanically or electrically scanning beams. The ultrasonic probe 12 contains therein the transducers (not shown) which serve as sources for generating ultrasonic waves and also receive reflection echoes.

Each transducer is generally formed to have a function of converting an input transmission signal of a pulse wave or continuous wave to an ultrasonic wave and emitting the ultrasonic wave, and a function of receiving an ultrasonic wave emitted from the inside of the object, converting the ultrasonic wave to a reception signal of an electrical signal and outputting the reception signal.

In general, an operation of pressing an object to acquire an elasticity image by using ultrasonic waves adopts a method of pressing the object through the ultrasonic probe 12 by an examiner in order to effectively apply a stress distribution to the body cavity of a diagnosis site of the object while transmitting/receiving ultrasonic waves by the ultrasonic probe 12. That is, a press plate is mounted on the ultrasonic wave transmitting/receiving face of the ultrasonic probe 12 so that the face of the press plate is fitted to the ultrasonic wave transmitting/receiving face, the press face constructed by the ultrasonic wave transmitting/receiving face of the ultrasonic probe 12 and the press plate is brought into contact with the body surface of the object, and the examiner manually moves the press face vertically to press the object.

The ultrasonic wave transmitting/receiving control circuit 18 controls the transmission and reception timings of ultrasonic waves. The transmitting circuit 14 generates a transmission pulse for driving the ultrasonic probe 12 to generate an ultrasonic wave, and also sets a convergence point of an ultrasonic wave transmitted from a built-in transmission phasing and summing circuit to some depth.

The receiving circuit 16 amplifies a reflection echo signal received by the ultrasonic probe 12 at a predetermined gain. Amplified reception signals whose number corresponds to the number of the transducers are input as respective independent reception signals to the phasing and summing circuit 20. The phasing and summing circuit 20 controls the phase of the reception signal amplified in the receiving circuit 16, and forms an ultrasonic beam at one point or plural convergence points.

The signal processing unit 22 receives the reception signal from the phasing and summing circuit 20, and executes various kinds of signal processing such as gain correction, log correction, wave detection, edge enhancement, or filter processing.

The ultrasonic probe 12, the ultrasonic wave transmitting/receiving control circuit 18, the transmitting circuit 14, the receiving circuit 16, the phasing and summing circuit 20 and the signal processing unit 22 constitute ultrasonic wave transmitting/receiving means, and an ultrasonic beam is scanned in a fixed direction in the body of the object by using the ultrasonic probe 12, thereby obtaining one tomographic image.

The monochromatic scan converter 26 is configured to contain cross-sectional scanning means for obtaining RF frame data of the inside of the object containing a moving tissue at an ultrasonic period by using the reflection echo signal output from the signal processing unit 22 of the ultrasonic wave transmitting/receiving means described above and means for controlling the system, for example, an A/D converter for converting the reflection echo signal from the signal processing unit 22 to a digital signal, plural frame memories for storing tomographic image data digitalized in the A/D converter in time-series, a controller for controlling the operation of the above units, etc.

The image display unit 24 displays the time-series tomographic image data obtained by the monochromatic scan converter 26, that is, a B-mode tomographic image, and it comprises a D/A converter for converting image data output from the monochromatic scan converter 26 through the switching and adding unit 42 to an analog signal, and a color television monitor for receiving an analog video signal from the D/A converter and displaying the analog video signal as an image.

The RF signal frame data selecting unit 28 undertakes a role of holding, into the frame memory provided thereto, the RF signal frame data which are successively output from the phasing and summing circuit 20 at the frame rate of the ultrasonic diagnostic apparatus with time (the RF signal frame data held at present is represented as RF signal frame data N), selecting one RF signal frame data (this is represented as RF signal frame data X) from timely past RF signal frame data N-1, N-2, N-3, . . . , N-M according to a control command of the ultrasonic diagnostic apparatus, and outputting a pair of RF signal frame data N and RF signal frame data X to the displacement measuring unit 30.

The signal output from the phasing and summing circuit 20 is described as the RF signal frame data. However, the signal may be a signal based on the I, Q signal format obtained by compositely demodulating the RF signal.

The displacement measuring unit 30 executes one-dimensional or two-dimensional correlation processing on the basis of a pair of RF signal frame data selected by the RF signal frame data selecting unit 28, measures a displacement or moving vector (the direction and magnitude of the displacement) of each measurement point on the tomographic image, and generates displacement frame data. A block matching method, a gradient method or the like is known as a method of detecting the moving vector, for example. According to the block matching method, an image is divided into blocks each of which comprises N×N pixels, for example, a block which is most approximate to a block of interest in the present frame is searched from a previous frame, and predictive coding is executed by referring to the searched block.

The distortion amount and elasticity modulus calculating circuit 32 calculates the distortion amount and the elasticity modulus of each measurement point on the tomographic image from the displacement frame data output from the displacement measuring unit 30 to generate numerical value data (elasticity frame data A) of the distortion amount or the elasticity modulus.

The switching and adding unit 42 serves as means for receiving the monochromatic tomographic image data from the monochromatic scan converter 26 and the elasticity image frame data output from the color scan converter 40, and adding or switching both the images, and it performs the switching operation so as to output only the monochromatic tomographic image data or only the color elasticity image data, or additionally combining and outputting both the image data.

Furthermore, as described in JP-A-2004-135929 which was previously filed by the applicant of this application, the color tomographic image may be displayed on the monochromatic tomographic image so as to be translucently superimposed on the monochromatic tomographic image. At this time, the monochromatic tomographic image is not limited to a general B-mode image, but may be a tissue harmonic tomographic image obtained by imaging harmonic components of a reception signal. Furthermore, in place of the monochromatic tomographic image, a tissue Doppler image may be displayed. As a modification example of the monochromatic tomographic image, at least one image may be a display target.

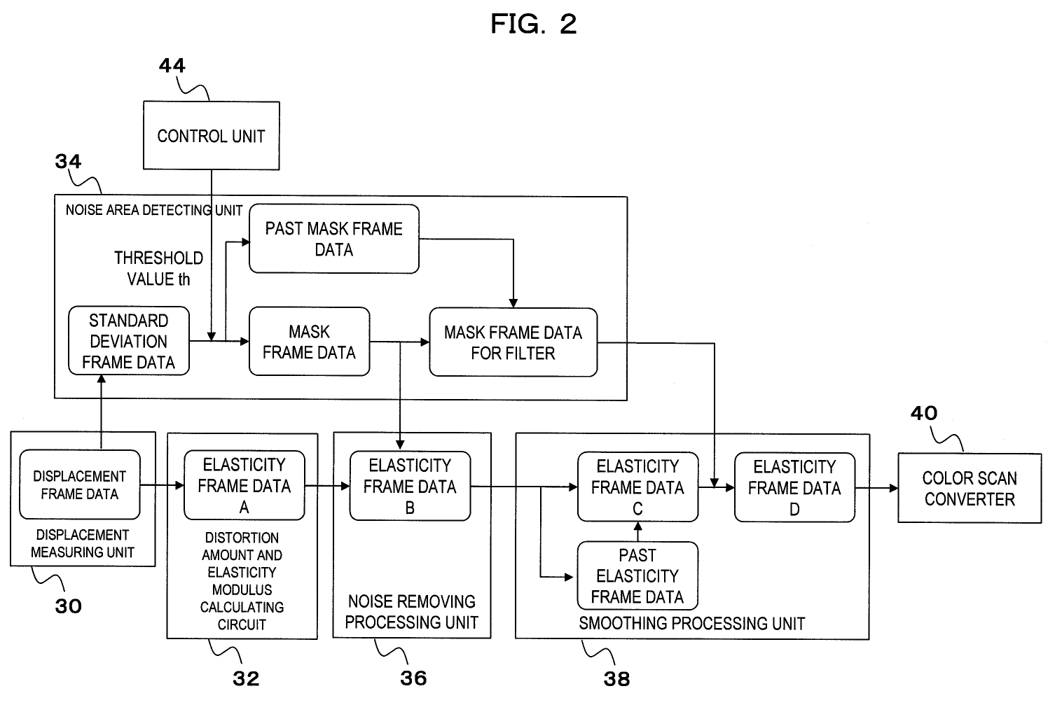

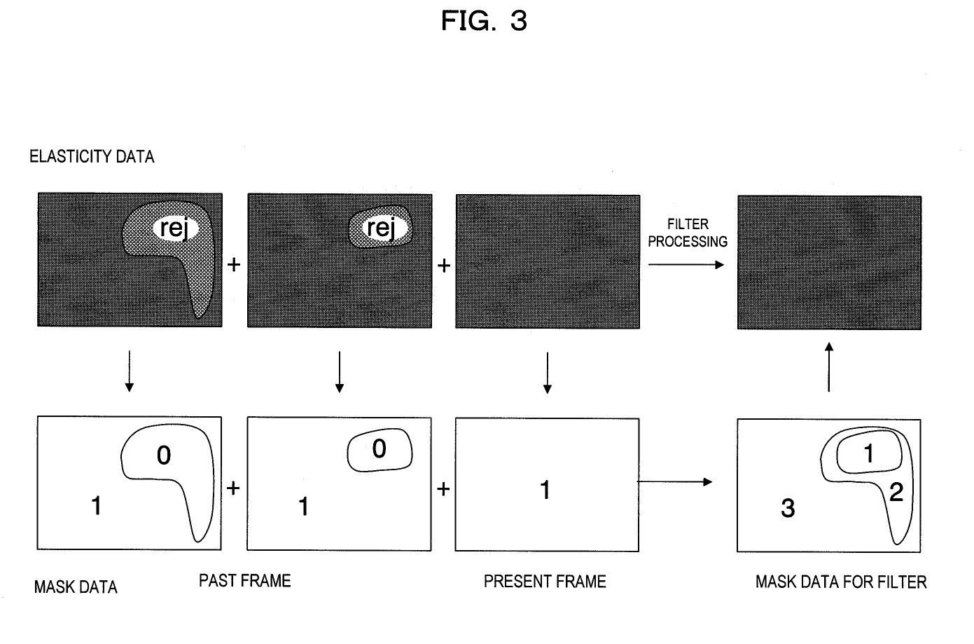

Subsequently, the noise area detecting unit 34, the noise removing processing unit 36 and the smoothing processing unit 38 as the feature portion of this embodiment will be described in detail with reference to FIGS. 2 and 3. The noise area detecting unit 34 first calculates standard deviation frame data from displacement frame data output from the displacement measuring unit 30. That is, the standard deviation is calculated with respect to the displacement information of plural measurement points which comprise a combination of the displacement information of some measurement point of interest of the displacement frame data and the displacement information of measurement points adjacent to the measurement point of interest, and the calculated standard deviation is set as a standard deviation at the measurement point of interest or at the plural measurement points.

This processing is executed on all the measurement points or representative measurement points while the measurement point of interest is successively moved, thereby calculating the standard deviation frame data. In place of the calculation of the standard deviation, a value representing the degree of dispersion of displacement information such as variance, a half-maximum full-width value of a displacement information distribution may be calculated.

Subsequently, mask frame data are generated by using the calculated standard deviation frame data and a threshold value th input from the controller 44. That is, when the standard deviation frame data is represented by SDi,j and the mask frame data is represented by Mi,j, the mask frame data is generated according to the following arithmetic expression.

When the standard deviation frame data SDi,j≧ the threshold value th, the mask frame data Mi,j=0 (i,j=1, 2, 3, . . . )

When the standard deviation frame data SDi,j<the threshold value th, the mask frame data Mi,j=1 (i,j=1, 2, 3, . . . )

As described above, the standard deviation at each measurement point of the standard deviation frame data is compared with the threshold value th and binarized. A measurement point which is larger than the threshold value th, in other words, when the displacements of plural measurement points containing a measurement point and measurement points adjacent to the measurement point concerned are dispersed, the measurement point concerned is set as a noise measurement point. A measurement point which is smaller than the threshold value this set as an adequate measurement point containing no noise. Accordingly, the mask frame data which are binarized into the noise measurement points and the adequate measurement points containing no noise are generated.

The noise removing processing unit 36 generates elasticity frame data B by using the elasticity frame data A output from the distortion amount and elasticity modulus calculating circuit 32 and the mask frame data output from the noise area detecting unit 34. That is, the elasticity information at the measurement points corresponding to “0” which is noise measurement points out of the respective measurement points of the elasticity frame data A is set to zero and removed (rejected) to thereby generate the elasticity frame data B. This processing is successively executed by using the mask frame data corresponding to the respective elasticity frame data A generated at different times.

The smoothing processing unit 38 executes filtering processing in the time direction by using the present elasticity frame data B and the elasticity frame data B which were generated in the past. An averaging filter, a Gaussian filter, a box filter or the like may be used for the processing method, and for example, FIG. 3 shows the processing concept when the averaging filter is used.

First, by using the respective mask frame data, the respective elasticity information at the corresponding measurement points of the past and present elasticity frame data B in which “0” is input to the elasticity frame data of the noise area is added to generate elasticity frame data C.

Subsequently, in order to average the elasticity frame data C, the elasticity frame data C is basically divided by the number of elasticity frame data as smoothing targets. However, in this case, the elasticity frame data C is not merely divided by the number of the elasticity frame data, but it is necessary to determine by using the mask frame data for filter generated in the noise area detecting unit 34 whether each measurement point is a noise area or not.

That is, in the noise area detecting unit 34, the mask data for filter are created from the present mask frame data and the past mask data which were generated in the past and are stored in the memory, and input into the smoothing processing unit 38. As shown in FIG. 3, the mask frame data for filter are frame data formed by summing the values of the measurement points corresponding to the present and past mask frame data. In this example, the mask frame data for filter are constructed by the respective values of “1”, “2” and “3”.

The smoothing processing unit 38 divides the elasticity information added to each measurement point of the elasticity frame data C by the value of the corresponding mask frame data for filter. That is, in the averaging operation, it is necessary to change the dividing value in consideration of how many frames of elasticity information are added in each area of the elasticity frame data. Therefore, the averaging is executed by using the mask data for filter output from the noise area detecting unit 34, and the elasticity frame data D is output.

The processing of the elasticity information according to the feature portion of the embodiment described above may be configured to be executed by a software program. That is, according to the ultrasonic diagnostic apparatus according to this embodiment, an ultrasonic elasticity information processing program which contains a step of determining the dispersion of plural displacement information containing displacement information of adjacent measurement points every displacement information of each measurement point of the generated displacement frame data, binarizing each determined dispersion on the basis of a predetermined threshold value, and detecting a noise area of the elasticity frame data, a step of removing the elasticity information of the detected noise area, and a step of smoothing the elasticity information of plural elasticity frame data generated at different times from which the elasticity information of the noise area is removed, in consideration of information as to whether each measurement point of each frame is a noise area or not are stored in a storage device, and arbitrarily executed.

The ultrasonic elasticity information processing program is not applied only to the ultrasonic diagnostic apparatus, but the ultrasonic elasticity information processing program described above may be stored and executed in an information processing device such as PC, for example. In this case, elasticity frame data obtained through an ultrasonic examination are input to the information processing device through an information recording medium or a network, and the noise removing processing, the smoothing processing, etc. may be executed.

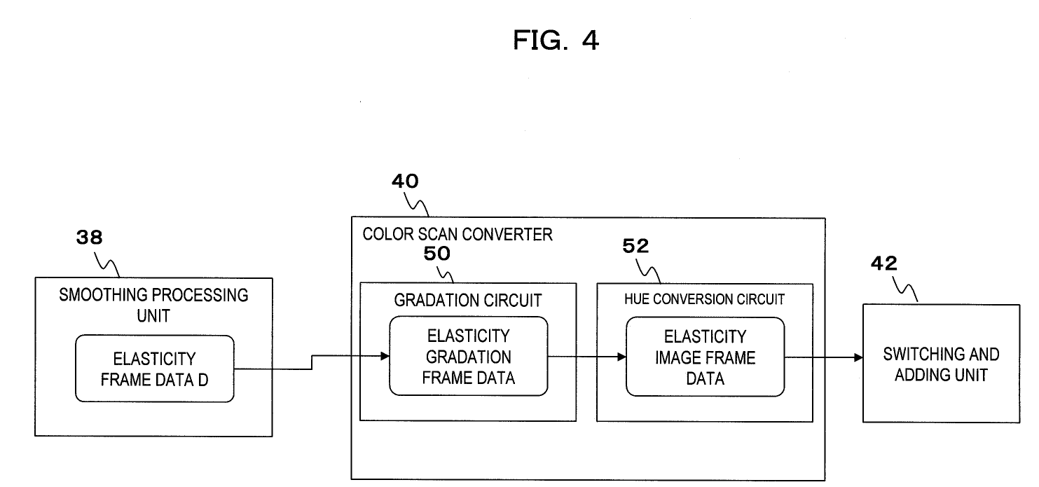

Next, an example of the operation of the color scan converter 40 will be described. The color scan converter comprises a gradation circuit 50 and a hue conversion circuit 52 as shown in FIG. 4. The color scan converter receives a command from the controller 44 or an upper limit value and a lower limit value defining a gradation selecting range in the elasticity frame data D output from the smoothing processing unit 38, and contains hue conversion processing of adding hue information of red, green, blue or the like as elasticity image data.

Furthermore, the color scan converter 40 may be a monochromatic scan converter 26. With respect to an area in which large distortion is measured, the brightness of the area in the elasticity image data is set to be higher, and with respect to an area in which small distortion is measured, the brightness of the area in the elasticity image data is set to be lower.

The gradation circuit 50 in the color scan converter 40 converts the elasticity frame data D by 255 steps in accordance with an instruction from the controller 44 or the magnitude of the value of element data of the elasticity frame data D in an area to be gradated, thereby generating elasticity gradation frame data. At this time, the area to be gradated is in a region of interest (ROI) set by the controller 44, however, the examiner arbitrarily changes the area concerned.

In the elasticity gradation frame data, with respect to the area in which large distortion is measured, the hue conversion circuit 52 in the color scan converter 40 converts the corresponding area in the elasticity image frame data to a red color, and conversely, with respect to the area where small distortion is measured, the corresponding area in the elasticity image frame data is converted to a blue code. Furthermore, when the measurement point of the elasticity gradation frame data is set to “0”, in other words, when all the areas corresponding to the elasticity frame data to be smoothed are noise areas and thus set to “0” (when the frame data for mask is also set to “0”), the corresponding area is converted to black.

As described above, according to the ultrasonic diagnostic apparatus according to this embodiment, the noise area is removed in each elasticity frame data, and thus the elasticity information of the noise area is not directly reflected to the finally generated elasticity image. Furthermore, plural elasticity frame data generated at different times from which the elasticity information of the noise areas is removed are smoothed in consideration of whether each measurement point of each elasticity frame data is a noise area or not, whereby the proper smoothing can be performed. As a result, the smoothed elasticity image can be stabilized and the image quality can be enhanced.

In this embodiment, the noise area detecting unit 34 generates the mask frame data, etc. on the basis of the displacement frame data input from the displacement measuring unit 30. However, the present invention is not limited to this style. For example, the mask frame data may be likewise generated on the basis of the elasticity frame data generated in the distortion amount and elasticity modulus calculating circuit 32.

That is, with respect to an area where a tissue is displaced by the press of the ultrasonic probe and a desired elasticity image is obtained, tissues are displaced all together to some extent by the press of the ultrasonic probe. On the other hand, with respect to a bloodstream area or the like, tissues are randomly displaced due to a factor other than the press of the probe. Therefore, it is possible to generate mask frame data on the basis of the information of the displacement, or even when elasticity information such as distortion or elasticity modulus calculated on the basis of the displacement is used, mask frame data can be likewise generated by detecting a noise area where tissues are randomly moved.

Furthermore, according to this embodiment, the noise area detecting unit 34 determines the standard deviation of each measurement point, and binarizes it on the basis of the threshold value th to generate the mask frame data, and the noise removing processing unit 36 sets “0” to the noise area. However, this embodiment is not limited to this style. For example, the noise area detecting unit 34 and the noise removing processing unit 36 may be configured as a weighting unit in combination so that the weighting unit performs weighting in accordance with the degree of sharpness of an elasticity information distribution of plural measurement points containing adjacent measurement points every elasticity information of each measurement point.

That is, as described above, in an area where a desired elasticity image is acquired, tissues are displaced all together to some extent by the press of the probe. Therefore, the frequency distribution of the elasticity information of that area is sharp, and thus the standard deviation thereof increases. On the other hand, in a bloodstream area or the like, tissues are randomly displaced and thus dispersed due to a factor other than the press of the probe, and thus the frequency distribution of the elasticity information of this area is flat and thus the standard deviation thereof is small. Accordingly, the degree of sharpness of the distribution of plural elasticity information is identified, and each elasticity information can be weighted in a multistage style in accordance with this identification.

For example, the weighting is more finely performed in accordance with not only the binarization, but the sharpness (flatness) degree of the distribution of the elasticity information (for example, the displacement), whereby it can be determined how appropriately the obtained elasticity information reflects the hardness or softness of the tissue.

Furthermore, the ultrasonic diagnostic apparatus according to this embodiment can be applied to a low echo area or an areas where the RF signal is unstable, for example to cyst, the inside of blood vessel, etc.

That is, The low echo area such as cyst has low SN, so that the displacement calculation precision is lowered and dispersion occurs in the displacement. When such an area is rejected, artifact is liable to occur at a boundary portion. Therefore, the image quality of the elasticity image can be enhanced by the correction. At this time, the smoothing processing of the smoothing processing unit 38 is executed by using the mask data for filter based on the noise area detecting unit 34 of the ultrasonic diagnostic apparatus according to this embodiment, whereby the image quality of the boundary portion can be enhanced and a stable image can be also acquired in the time direction.

Furthermore, as described above, the RF signal is random and also a calculation error is liable to occur in a blood-streaming area in a blood vessel, and thus it is desired to remove the blood-streaming area when an elasticity image of blood clot is constructed.

The detection of the bloodstream area can be performed by calculating a value representing displacement dispersion such as variance or standard deviation of the distribution of displacement frame data and comparing this value with a threshold value. The value of a correlation coefficient representing the degree of correlation in the correlation calculation when the displacement is calculated is low in such a bloodstream area, and thus the bloodstream area can be detected on the basis of the correlation coefficient.

The detection of the bloodstream area based on the correlation coefficient may be performed by determining the correlation coefficient every measurement point to generate correlation coefficient frame data, calculating the variance of plural correlation coefficients containing the correlation coefficients of adjacent measurement points every measurement point, comparing the calculated variance with a threshold value to perform sectionalization, and detecting as the contour of the bloodstream area a place which is different in section from the adjacent measurement points.

According to the method as described above, the bloodstream (noise area) is detected from each elasticity frame data and removed, and the smoothing processing of the smoothing processing unit 38 using the mask data for filter based on the noise area detecting unit 34 of the ultrasonic diagnostic apparatus of this embodiment is executed, whereby the image quality of the elasticity image containing the bloodstream area can be enhanced.

1. An ultrasonic diagnostic apparatus characterized by comprising:
an ultrasonic probe configured to transmit/receive an ultrasonic wave to/from an object to be examined; a phasing and summing unit configured to generate RF signal frame data of a cross-sectional site of the object on the basis of a reflection echo signal measured by the ultrasonic probe; an elasticity calculator configured to generate frame data of elasticity information representing the degree of hardness or softness of a tissue at each measurement point of the cross-sectional site on the basis of a pair of RF signal frame data acquired in different times; a weighting unit configured to perform weighting on the elasticity information of each measurement point of the generated elasticity frame data in accordance with the degree of sharpness of a distribution of elasticity information for plural measurement points containing adjacent measurement points; a smoothing unit for smoothing the weighted elasticity information of the plurality of elasticity frame data generated at the different times; and an elasticity image display unit for generating an elasticity image on the basis of the smoothed elasticity frame data and displaying the generated elasticity image on a display unit.

2. The ultrasonic diagnostic apparatus according to claim 1, wherein the weighting unit comprises a noise area detecting unit for binarizing the degree of sharpness of distribution of the elasticity information of the plural measurement points on the basis of a predetermined threshold value and detecting a noise area of the elasticity frame data, and a noise removing unit for removing elasticity information of the detected noise area.

3. The ultrasonic diagnostic apparatus according to claim 1, wherein the smoothing unit smoothes the weighted elasticity information of the plurality of elasticity frame data generated at different times on the basis of weighting degree of the elasticity information of each measurement point.

4. The ultrasonic diagnostic apparatus according to claim 1, wherein the smoothing unit identifies on the basis of mask frame data for filter generated by the noise area detecting unit whether each measurement point is a noise area or not, and divides the elasticity frame data by the number of elasticity frame data to be smoothed in order to average the elasticity frame data, whereby the elasticity frame data are averaged.

5. The ultrasonic diagnostic apparatus according to claim 1, wherein RF signal frame data of a cross-sectional site of the object is at least one of a B-mode image, a tissue harmonic tomographic image and a tissue Doppler image.

6. An ultrasonic elasticity information processing method for smoothing, between elasticity frame data generated at different times, frame data of elasticity information representing the degree of hardness or softness of a measurement point of a tissue at the measurement point of a cross-sectional site of an object which is generated from a pair of RF signal frame data acquired at different times based on reflection echo signals of the cross-sectional site of the object measured by transmitting/receiving ultrasonic waves to/from the object and displaying the smoothed frame data on a display unit, characterized by comprising:
a step of weighting elasticity information of each measurement point of the generated elasticity frame data in accordance with sharpness degree of elasticity information of plural measurement points containing adjacent measurement points; and a step of smoothing elasticity information of plural weighted elasticity frame data generated at different times.

7. The ultrasonic elasticity information processing method according to claim 6, wherein the weighting step comprises a step for binarizing the degree of sharpness of the elasticity information of the plural measurement points on the basis of a predetermined threshold value and detecting a noise area of the elasticity frame data, and a step for removing elasticity information of the detected noise area.

8. The ultrasonic elasticity information processing method according to claim 6, wherein smoothing of elasticity information of a plurality of elasticity frame data generated at different times is executed by smoothing the plurality of weighted elasticity frame data generated at different times on the basis of weighting degree of the elasticity information of each measurement point.

9. The ultrasonic elasticity information processing method according to claim 6, wherein the smoothing unit identifies whether each measurement point is a noise area or not on the basis of mask frame data for filter generated by the noise area detecting unit, and divides the elasticity frame data by the number of elasticity frame data to be smoothed in order to average the elasticity frame data, whereby the elasticity frame data are averaged.

10. The ultrasonic elasticity information processing method according to claim 6, wherein RF signal frame data of a cross-sectional site of the object is at least one of a B-mode image, a tissue harmonic tomographic image and a tissue Doppler image.

11.-15. (canceled)

16. An ultrasonic diagnostic apparatus characterized by comprising:
an ultrasonic probe configured to transmit/receive an ultrasonic wave to/from an object;
an elasticity calculating unit configured to generate an elasticity frame data representing the degree of hardness or softness of a tissue on the basis of a reflection echo signal measured by the ultrasonic probe;
a noise area detecting unit configured to detect a noise area of the elasticity frame data; a noise removing unit configured to remove elasticity information of the noise area from the elasticity frame data;
a smoothing unit configured to smooth the plurality of the elasticity frame data from which elasticity information of the noise area are removed; and
a display unit configured to display an elasticity image based on the smoothed elasticity frame data.

17. The ultrasonic diagnostic apparatus according to claim 16, wherein the smoothing unit smoothes the elasticity frame data by averaging the elasticity information added at each measurement point of the elasticity frame data.

18. The ultrasonic diagnostic apparatus according to claim 16, wherein the smoothing unit identifies whether each measurement point is a noise area or not on the basis of mask frame data for filter generated by the noise area detecting unit, and divides the elasticity frame data by the number of elasticity frame data to be smoothed in order to average the elasticity frame data, whereby the elasticity frame data are averaged.

19. The ultrasonic diagnostic apparatus according to claim 16, wherein the noise area detecting unit detects the noise area of the elasticity frame data by binarizing distribution of the elasticity information at each of the plural measurement points on the basis of a predetermined threshold value.

20. The ultrasonic diagnostic apparatus according to claim 16, characterized in comprising a weighting unit configured to perform weighting on elasticity information of each measurement point of the generated elasticity frame data in accordance with distribution of elasticity information for plural measurement points including the adjacent measurement points.

21. The ultrasonic diagnostic apparatus according to claim 20, wherein the smoothing unit smoothes the weighted elasticity information of the plurality of elasticity frame data generated at different times on the basis of weighting degree of the elasticity information of each measurement point.

22. The ultrasonic diagnostic apparatus according to claim 20, wherein the weighting unit performs weighting on elasticity information at each measurement point of the generated elasticity frame data in accordance with the degree of sharpness of distribution of elasticity information for plural measurement points containing adjacent measurement points.

23. An ultrasonic elasticity information processing method comprising:
transmitting/receiving an ultrasonic wave to/from an object;
generating elasticity frame data representing the degree of hardness or softness of a tissue on the basis of a reflection echo signal measured by the ultrasonic probe;
detecting a noise area of the elasticity frame data;
removing elasticity information of the noise area from the elasticity frame data;
smoothing the plurality of the elasticity frame data from which elasticity information of the noise area is removed; and
displaying an elasticity image based on of the smoothed elasticity frame data.
